(19) United States
(12) Patent Application Publication
(10) Pub. No.: US 2024/0408178 A1
(43) Pub. Date: Dec. 12, 2024

(54) LOW-DOSE INTERFERON COMPOSITION FOR CAT WITH LOW ADVERSE EFFECTS AND ITS USE AND METHOD

(71) Applicant: Ainos, Inc., San Diego, CA (US)

(72) Inventors: Tsung-Fu YU, New Taipei City (TW); Chun-Hsien TSAI, New Taipei City (TW); Ting-Chuan LEE, New Taipei City (TW)

(21) Appl. No.: 18/731,677

(22) Filed: Jun. 3, 2024

(30) Foreign Application Priority Data
Jun. 6, 2023 (TW) 112121122

(51) Int. Cl. A61K 38/21; A61K 9/00

(52) U.S. Cl. CPC A61K 38/212; A61K 9/006

(57) ABSTRACT

Provided is a low-dose interferon composition for treatment or prevention of conditions, diseases or disorders associated with inflammation in cat. The composition comprises a total concentration of human interferon-α2b in a range of 160 IU/ml to 100,000 IU/ml. The composition is orally administered buccally.

### CROSS-REFERENCE TO RELATED APPLICATION

The present application claims priority to Taiwan Patent App. No. 112121122, filed Jun. 6, 2023, the entirety of which are incorporated by reference herein

### FIELD OF THE INVENTION

The present disclosure relates to a composition containing low-dose interferon for cat, with low adverse effects, which is useful for treatment or prevention of conditions, diseases or disorders associated with inflammation. The present disclosure also relates to a use and a method of the composition to treat conditions, diseases or disorders of cat associated with inflammation.

### BACKGROUND OF THE INVENTION

Interferon (IFNs) allow for communication between cells to trigger the protective defenses of the immune system that eradicate pathogens or tumors. When an organism is infected by a virus, the infected cells will release interferon, then the uninfected cells nearby receive the released interferon and increase the synthesis of antiviral proteins, thereby interfering with viral replication and preventing further infection. Because interferon can induce an immune response in organisms, it has been commercially used to treat diseases in cats and dogs, such as oral lesions, relieving pains, improving appetite, skin diseases, etc.

As far as feline interferons are concerned, the most widely commercially available product is recombinant feline interferon omega (rFeIFN-ω). However, this feline interferon is not only expensive, but also has the side effects of hyperthermia and leukopenia after administration. Therefore, reducing the side effects while effectively achieving the therapeutic effect is an urgent problem that needs to be solved.

### SUMMARY OF THE INVENTION

The present invention provides, in part, a low-dose interferon-containing composition, for treatment or prevention of conditions, diseases or disorders associated with inflammation in cat, the composition comprises a total concentration of human interferon-α2b in a range of 160 IU/ml to 100,000 IU/ml.

The present invention also provides, in part, a use of the composition in the manufacture of a medicament for the treatment or prevention of conditions, diseases or disorders associated with inflammation in cat.

The present invention also provides, in part, a method for treatment or prevention of conditions, diseases or disorders associated with inflammation in cat, the method comprises a step of orally administering to a cat subject of a low-dose interferon-containing composition in a daily dose of 160 IU/ml to 100,000 IU/ml human interferon-α2b.

### BRIEF DESCRIPTION OF THE DRAWINGS

FIG. 1 shows the microscopic images of cells in the cell control group, the virus control group and the experimental examples which were administered at concentrations of 100,000 IU/ml, 20,000 IU/ml, 4,000 IU/ml, 800 IU/ml, 160 IU/ml, 32 IU/ml, 6.4 IU/ml, 1.28 IU/ml, 0.25 IU/ml, and 0.0512 IU/ml;

FIG. 2 shows the microscopic images of cells in the cell control group, the virus control group and the comparative examples which were administered at concentrations of 100,000 IU/ml, 20,000 IU/ml, 4,000 IU/ml, 800 IU/ml, 160 IU/ml, 32 IU/ml, 6.4 IU/ml, 1.28 IU/ml, 0.25 IU/ml, and 0.0512 IU/ml;

FIG. 3 shows the microscopic images of cells cultured for 3 days in the medium control group, solvent control and the experimental examples which were administered at concentrations of 0.05 MIU/ml (50,000 IU/ml), 0.025 MIU/ml (25,000 IU/ml), 0.0125 MIU/ml (12,500 IU/ml), 0.00625 MIU/ml (6,250 IU/ml), 0.00313 MIU/ml (3,130 IU/ml), 0.00156 MIU/ml (1,560 IU/ml), 0.00078 MIU/ml (780 IU/ml), and 0.00039 MIU/ml (390 IU/ml); and

FIG. 4 shows the microscopic images of cells cultured for 6 days in the medium control group, solvent control and the experimental examples which were administered at concentrations of 0.05 MIU/ml (50,000 IU/ml), 0.025 MIU/ml (25,000 IU/ml), 0.0125 MIU/ml (12,500 IU/ml), 0.00625 MIU/ml (6,250 IU/ml), 0.00313 MIU/ml (3,130 IU/ml), 0.00156 MIU/ml (1,560 IU/ml), 0.00078 MIU/ml (780 IU/ml), and 0.00039 MIU/ml (390 IU/ml).

### DETAILED DESCRIPTION OF THE PREFERRED EMBODIMENTS

As used herein, the term “treating” or “treatment” includes prophylaxis of the specific disorder or condition, or alleviation of the symptoms associated with a specific disorder or condition and/or preventing or eliminating said symptoms.

As used herein, the term “prevention” or any grammatical variation thereof (e.g., prevent, preventing, and prevention etc.), as used herein, includes but is not limited to, delaying the onset of symptoms, preventing relapse to a disease, increasing latency between symptomatic episodes, or a combination thereof. Prevention, as used herein, does not require the complete absence of symptoms.

As used herein, the terms “comprises,” “comprising,” “includes,” “including,” “has,” “having,” “contains”, “containing,” or any other variation thereof, are intended to encompass a non-exclusive inclusion, subject to any limitation explicitly indicated otherwise, of the recited components. For example, a composition, and/or a method that “comprises” a list of elements (e.g., components, features, or steps) is not necessarily limited to only those elements (or components or steps) but may include other elements (or components or steps) not expressly listed or inherent to the composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure, or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The numerical range defined in the present invention is continuous and, unless otherwise specified, includes all values from the minimum value to the maximum value of the range, and the numerical values used in the present invention to express the component content and reaction conditions are approximate values and are to reflect the factors. Reasonable errors arising from experiments or measurements.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

A low-dose interferon-containing composition is provided herein for treatment or prevention of conditions, diseases or disorders associated with inflammation in cat. In one aspect, the low-dose interferon-containing composition is a low-dose human interferon-α2b containing composition for treatment or prevention of infection of Felis catus whole fetus cells (Fcwf-4) in cats caused by virus.

In an example, the interferon is a human interferon-α2b. The composition comprises a total concentration of human interferon-α2b in a range of 160 IU/ml to 100,000 IU/ml. The human interferon-α2b may be diluted by buffers, solvents or other compositions to the above-mentioned concentration. The human interferon-α2b is interferon of human origin and may be a recombinant interferon-α2b of human origin. The composition may be administered as an oral formulation to a desired feline subject, allowing pet owners to conveniently feed and administer it at home. This contrasts with the commercially available interferon product, Virbagen Omega, which requires pet owners to bring their cats to a clinic for injections.

In an example, the composition contains only one type (or class) of human interferon. Namely, the composition does not contain any interferon other than the human interferon-α2b. In an example, the composition is consisted of a total concentration of human interferon-α2b in a range of 160 IU/ml to 100,000 IU/ml and one or more additives other than interferon.

In different examples, the concentration of the interferon in the composition may be 160 IU/ml or above, 200 IU/ml or above, 300 IU/ml or above, 400 IU/ml or above, 500 IU/ml or above, 600 IU/ml or above, 700 IU/ml and above, above 800 IU/ml, above 900 IU/ml, above 1,000 IU/ml, above 1,100 IU/ml; and below 100,000 IU/ml, below 80,000 IU/ml, below 60,000 IU/ml, 50,000 IU/ml or less, 40,000 IU/ml or less, 30,000 IU/ml or less, 20,000 IU/ml or less, 15,000 IU/ml or less, 12,000 IU/ml or less, 11,500 IU/ml or less, 11,000 IU/ml or less, 10,500 IU/ml or less; below 10,000 IU/ml, below 9,500 IU/ml, below 9,000 IU/ml, below 8,500 IU/ml, below 8,000 IU/ml, below 7,500 IU/ml, below 7,000 IU/ml, below 6,500 IU/ml, 6,000 Below IU/ml, below 5,500 IU/ml, below 5,000 IU/ml, below 4,500 IU/ml, below 4,000 IU/ml, below 3,500 IU/ml, below 3,000 IU/ml, below 2,500 IU/ml, below 2,000 IU/ml or less, 1,500 IU/ml or less.

In some examples, the concentration of the interferon may be 200 IU/ml to 1,1000 IU/ml, 500 IU/ml to 5,000 IU/ml, or 700 IU/ml to 1,500 IU/ml. In some examples, the concentration of the interferon can be about 820 IU/ml, 1,062 IU/ml, 10,550 IU/ml.

In one aspect, the composition of the present invention is pharmaceutical compositions or therapeutic compositions, which includes but is not limited to being administered to a desired subject by subcutaneous or intramuscular injection, application or oral administration. The subject is cat. In one aspect, the cats may be nursing cats. In one aspect, the composition is administered to a subject in an oral formulation, which may be a pharmaceutically acceptable solid dosage form or a pharmaceutically acceptable liquid dosage form. In one embodiment, the solid dosage form is a saliva-soluble solid dosage form, such as an orally dissolvable tablet. In this embodiment, the composition further includes one or more pharmaceutically acceptable additives, which may be used as excipients, antioxidants, chelating agents, binders, lubricants and/or surfactants as needed. The additive includes but is not limited to cellulose, starch, corn starch, white sugar, lactose, glucose, sorbitol, mannitol, maltose powder, lactose, silica, malic acid or a combination of at least two of the above.

In one example, the composition contains one or more additives, and the content of the additives may account for 0.1% to 99.9% of the total weight of the composition, and in the composition containing one or more additives, the interferon concentrations range from 160 IU/ml to 100,000 IU/ml.

In one embodiment, the composition includes maltose powder, silica and malic acid, the maltose powder is used as an excipient, and the weight of the maltose powder accounts for between 90.00% and 99.50% of the total weight of the composition. The weight of the silica accounts for the total weight of the composition between 0.1% and 2.0%, and the weight of the malic acid accounts for the total weight of the composition between 0.01% and 9.50%. In the composition, the concentration of human interferon-α2b ranges from 600 IU/ml to 1,200 IU/ml.

In one aspect, the diseases for which the composition of the present invention is used to treat or prevent include, but are not limited to, diseases caused by canine parvovirus, equine herpesvirus, feline coronavirus, feline distemper virus, feline leukemia virus, feline HIV, and feline infectious diseases. Disease caused by peritonitis virus, feline calisivirus, feline herpes virus, or other similar viruses.

On the other hand, the diseases used to treat or prevent the composition of the present invention include but are not limited to dermatitis, upper respiratory tract infection, oral diseases (such as gingivitis, periodontal disease), etc.

In one aspect, the composition of the present invention may be formulated to form food compositions, immunity-enhancing food compositions, health supplements, feed compositions for companion animals, a variety of pharmaceutical compositions, and the like. For example, the composition may be formulated to form a food product such as frozen food, canned food, dairy products, fermented products, gel food, dental candy, or jerky for pets, etc.

The present invention provides a composition for preparing a pharmaceutical composition for treating diseases caused by viral infection in cats. The composition contains a low dose of interferon, and the concentration of the interferon is 160 IU/ml to 100,000 IU/ml.

The following examples are provided to illustrate the present invention. It should be understood, however, that the invention is not to be limited to the specific conditions or details described in these examples.

### Antiviral Activity Test

Felis catus whole fetus cells (Fcwf-4) were used as the cells to be infected, and feline coronavirus (FCoV) was used as the infecting virus. The interferon was administered to the infected cells to compare the effect of the composition of the present invention (experimental examples) with a commercially available interferon (comparative examples). The interferon used in the experimental example is human interferon-α2b according to the present invention, while the interferon used in the comparative example is the commercially available Virbagen Omega, which is recombinant interferon omega of feline origin that is used to treat a range of viral diseases in cats and dogs. The purpose of this test is to verify that the low-dose human interferon α-2b of the present invention can exert immunomodulatory or antiviral effects on cat cells.

In the cell preparation, the Fcwf-4 were seeded in a 96-well plate at a cell concentration of 1×10\hat{ }4 cells per well and cultured for 24 hours. The culture was carried out in a CO₂ incubator (37° C., 5% CO₂).

The experimental examples and the comparative examples are treatment groups. The interferon was added to the culture medium of Fcwf-4 at a concentration of 0.1 ml per well. After addition, the culture was continued for 24 hours. Then the Fcwf-4 contained culture medium was removed and replaced with a fresh medium. Each group was repeated 6 times.

The original concentration of the interferon for both experimental examples and comparative examples is 10\hat{ }7 IU/ml, which is prepared by serial dilution to the following concentrations: 100,000 IU/ml, 20,000 IU/ml, 4,000 IU/ml, 800 IU/ml, 160 IU/ml, 32 IU/ml, 6.4 IU/ml, 1.28 IU/ml, 0.25 IU/ml, 0.0512 IU/ml.

After the culture is completed, the feline coronavirus (FCoV) at a concentration of 100 TCID50 (tissue culture infectious dose) was added to the culture medium. Then, the culture medium containing the feline coronavirus was incubated at 37° C. for 2 hours to allow the Fcwf-4 cells to be infected by the feline coronavirus.

For comparison purposes, a cell control group and a virus control group were also prepared. In the cell control group, no feline coronavirus or interferon was added. In the virus control group, the feline coronavirus was added, but no interferon was administered. The culture time for both the cell control group and the virus control group was 42 hours. Then the Fcwf-4 contained culture medium was removed and replaced with a fresh medium.

For the experimental examples, comparative examples, cell control group and virus control group, the cell lesions are observed under the microscope, and the cell viability was assessed by the neutral red uptake assay, and the absorbance (Optical Density, OD) at a wavelength of 540 nm is measured by the ELISA instrument. The 100% viability was set by the OD mean of the cell control group and 0% viability was set by the OD mean of the virus control group to normalize and calculate the relative viability for each. The results are shown in Table 1.

In addition to measuring and comparing the OD values of the experimental examples and the comparative examples to determine the cell viability, the concentration for 50% of maximal effect (EC50) was calculated by using GraphPad Prism 8.0.

Table 2 shows the results of the absorbance (Optical Density, OD) and the cell viability of the experimental examples and the comparative examples. The EC50 in the experimental examples is approximately 1,062 IU/ml, and the effective dose in the experimental examples is approximately 5.29 ng/ml.

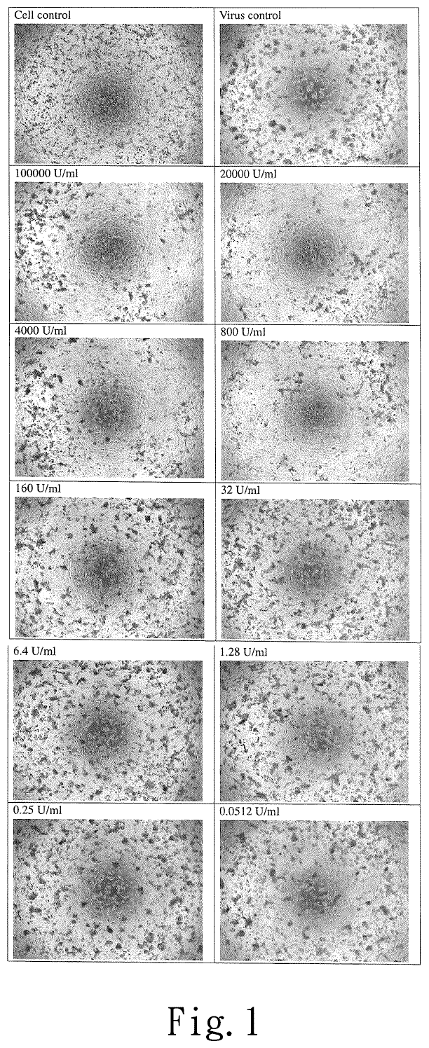

FIG. 1 shows the microscopic images of cells in the cell control group, the virus control group and the experimental examples which were administered at concentrations of 100,000 IU/ml, 20,000 IU/ml, 4,000 IU/ml, 800 IU/ml, 160 IU/ml, 32 IU/ml, 6.4 IU/ml, 1.28 IU/ml, 0.25 IU/ml, and 0.0512 IU/ml.

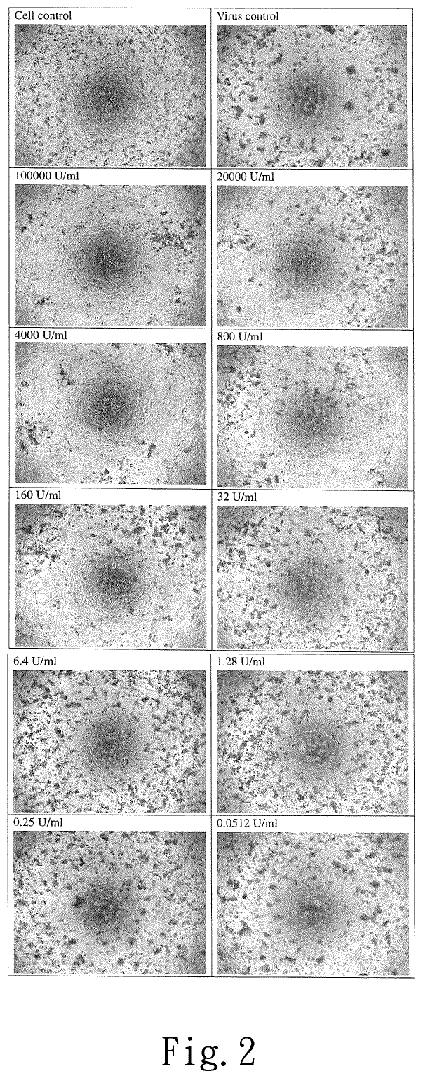

FIG. 2 shows the microscopic images of cells in the cell control group, the virus control group and the comparative examples which were administered at concentrations of 100,000 IU/ml, 20,000 IU/ml, 4,000 IU/ml, 800 IU/ml, 160 IU/ml, 32 IU/ml, 6.4 IU/ml, 1.28 IU/ml, 0.25 IU/ml, and 0.0512 IU/ml.

It can be seen from the microscopic photos that after administration of low-dose human interferon-α2b of the present invention, the proportion of cell lesions is reduced compared with the virus control group that is not administered. There is a difference of about 80% in the number of cells with cytotoxicity between the virus control group and the experimental example (800 IU/ml). The experimental example shows that the composition of the present invention can effectively reduce the proportion of cells infected by viruses and increase the survival rate of infected cells.

### Cytotoxicity Analysis

Felis catus whole fetus cells (Fcwf-4) were used as the cells to be infected, and feline coronavirus (FCoV) was used as the infecting virus. The interferon was administered to the infected cells to compare the effect of the composition of the present invention (experimental examples) with a commercially available interferon (comparative examples). The interferon used in the experimental example is human interferon-β2b according to the present invention, while the interferon used in the comparative example is the commercially available Virbagen Omega. The purpose of this analysis is to determine the cytotoxicity of the human interferon-α2b-containing composition for cats, in order to verify its safety. In the cell preparation, the Fcwf-4 were seeded in a 96-well plate at a cell concentration of 1×10\hat{ }4 cells per well and cultured for 24 hours. The culture was carried out in a CO₂ incubator (37° C., 5% CO₂).

The original concentration of the interferon for both experimental examples and comparative examples is 10\hat{ }7 IU/ml, which is prepared by serial dilution to the following concentrations: 100,000 IU/ml, 20,000 IU/ml, 4,000 IU/ml, 800 IU/ml, 160 IU/ml, 32 IU/ml, 6.4 IU/ml, 1.28 IU/ml, 0.25 IU/ml, 0.0512 IU/ml.

The experimental examples and the comparative examples are treatment groups. Various concentrations of the interferon were added to the Fcwf-4 contained culture medium and cultured for 68 hours.

Then the Fcwf-4 contained culture medium was removed and replaced with a fresh medium. After removing, MTS/PMS solution was added into the medium. The culture was carried out in a CO₂ incubator (37° C., 5% CO₂) for 24 hours. The supernatants of the medium were then collected for ELISA analysis at a wavelength of 490 nm.

Table 3 shows the measured absorbance and cell viability. From the results, the cell viability is 85% when using human interferon-(2b at a concentration of 4,000 IU/ml, and the cell viability is close to 90% when using human interferon-α2b at a concentration of 800 IU/ml. It can be seen that within the dose range of the present invention, the proportion of cat cells infected by viruses can be effectively reduced, adverse effects to cat cells are minimized, and toxicity to cat cells is low.

In addition, at each concentration higher than 6.4 IU/ml, the cell viability of the experimental example is higher than that of the comparative example, showing that the adverse effects caused by the composition of the present invention are lower than the commercially available cat interferon.

### MTT Cell Viability Analysis

In the experimental examples, Felis catus whole fetus cells (Fcwf-4) were administered with the human interferon-α2b according to the present invention. The purpose of this analysis is to determine the cytotoxicity of the human interferon-α2b-containing composition for cats to verify its safety.

In the cell preparation, the Fcwf-4 were seeded in a 96-well plate at a cell concentration of 1×10\hat{ }3 cells per well and cultured for 22±2 hours. The culture was carried out in a CO₂ incubator (37° C., 5% CO₂).

The interferon was added to the culture medium of Fcwf-4 at a concentration of 0.2 ml per well. After addition, the culture was continued for 3 days and 6 days, separately. Then the Fcwf-4 contained culture medium was removed and replaced with a fresh medium. Each group was repeated 4 times.

The interferon was diluted and prepared to the following concentrations: 0.05 MIU/ml (50,000 IU/ml), 0.025 MIU/ml (25,000 IU/ml), 0.0125 MIU/ml (12,500 IU/ml), 0.00625 MIU/ml (6,250 IU/ml) ml), 0.00313 MIU/ml (3,130 IU/ml), 0.00156 MIU/ml (1,560 IU/ml), 0.00078 MIU/ml (780 IU/ml), 0.00039 MIU/ml (390 IU/ml), MIU is 10\hat{ }6 IU.

MTT solution was added into the medium at a concentration of 0.2 ml per well. The culture was carried out in a CO₂ incubator (37° C., 5% CO₂) for 3±0.1 hours, to form purple precipitates. The incubation was terminated through the solution disposal. The isopropanol at 0.1 ml/well was added and shaken for 10 minutes to dissolve the precipitate for ELISA reading under 570 nm (OD).

For comparison purposes, a medium control group and a solvent control group are also prepared. The control group contains the Fcwf-4 without any interferon added. The buffer concentration of the medium control group is 0% and the buffer concentration of the solvent control group is 7%. The 100% viability was set by the OD mean of the medium control group to normalize and calculate the relative viability for each example. The half maximal inhibitory concentration (IC50) was calculated by using GraphPad Prism 8.0 GraphPad Prism 8.0.

Table 4 shows the measured absorbance and the MTT cell viability assay after culture for 3 days and 6 days, respectively. According to the results, after 6 days, the cells administered with the human interferon-(2b according to the present invention have a cell viability of more than 50%. In addition, the IC50 is approximately 10,550 IU/ml, and the effective dose in the experimental examples is approximately 59.825 ng/ml.

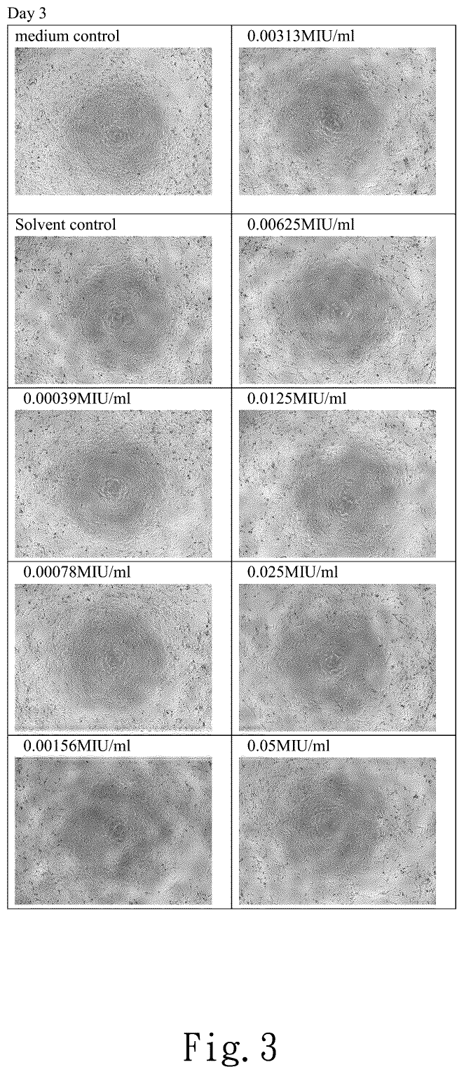

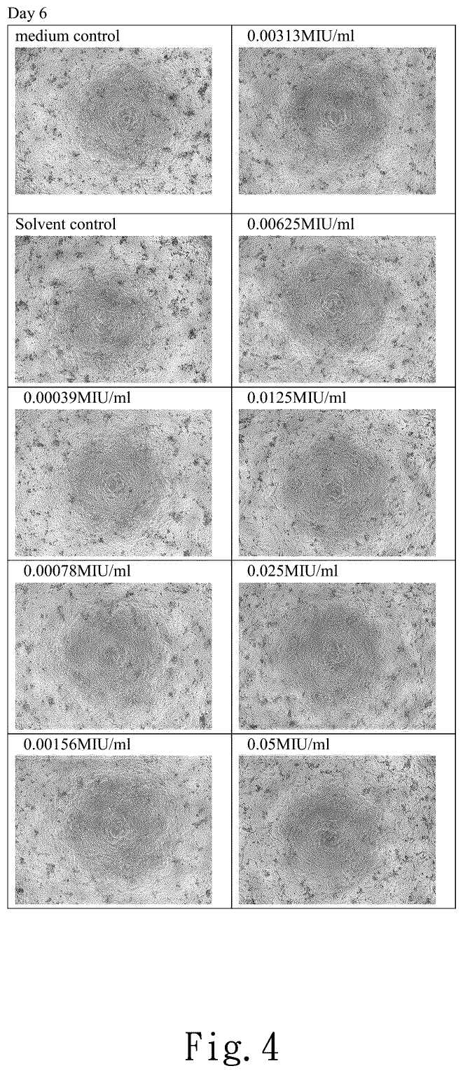

FIG. 3 and FIG. 4 show the microscopic images of cells in the medium control group, solvent control and the experimental examples which were administered at concentrations of 0.05 MIU/ml (50,000 IU/ml), 0.025 MIU/ml (25,000 IU/ml), 0.0125 MIU/ml (12,500 IU/ml), 0.00625 MIU/ml (6,250 IU/ml), 0.00313 MIU/ml (3,130 IU/ml), 0.00156 MIU/ml (1,560 IU/ml), 0.00078 MIU/ml (780 IU/ml), and 0.00039 MIU/ml (390 IU/ml).

TABLE 1

| | | Cell | | Cell |
|---|---|---|---|---|
| | OD | viability (%) | OD | viability (%) |
| Cell control group | 1.070 ± 0.017 | 100 ± 2.49 | 1.078 ± 0.007 | 100 ± 0.98 |
| Virus control group | 0.378 ± 0.023 | 0 ± 3.38 | 0.340 ± 0.016 | 0 ± 2.18 |

TABLE 2

| | Experimental examples | | Comparative examples | |
|---|---|---|---|---|
| Concentration | | Cell | | Cell |
| (IU/ml) | OD | viability (%) | OD | viability (%) |
| 100,000 | 0.885 ± 0.078 | 73.34 ± 11.31 | 0.845 ± 0.072 | 68.46 ± 9.78 |
| 20,000 | 0.825 ± 0.080 | 64.57 ± 11.52 | 0.907 ± 0.044 | 76.92 ± 6.04 |
| 4,000 | 0.823 ± 0.049 | 64.40 ± 7.05 | 0.886 ± 0.063 | 74.02 ± 8.60 |
| 800 | 0.759 ± 0.067 | 55.13 ± 9.66 | 0.927 ± 0.032 | 79.58 ± 4.40 |
| 160 | 0.603 ± 0.040 | 32.59 ± 5.82 | 0.886 ± 0.050 | 73.98 ± 6.81 |
| 32 | 0.550 ± 0.027 | 24.83 ± 3.93 | 0.737 ± 0.047 | 53.81 ± 6.35 |
| 6.4 | 0.523 ± 0.008 | 20.98 ± 1.18 | 0.514 ± 0.013 | 23.60 ± 1.83 |
| 1.28 | 0.492 ± 0.032 | 16.55 ± 4.66 | 0.484 ± 0.026 | 19.44 ± 3.54 |
| 0.25 | 0.450 ± 0.023 | 10.45 ± 3.39 | 0.452 ± 0.027 | 15.17 ± 3.67 |
| 0.0512 | 0.466 ± 0.019 | 12.72 ± 2.80 | 0.461 ± 0.024 | 16.35 ± 3.23 |

TABLE 3

| | Experimental examples | | Comparative examples | |
|---|---|---|---|---|
| Concentration | | Cell | | Cell |
| (IU/ml) | OD | viability (%) | OD | viability (%) |
| 0 | 1.397 ± 0.019 | 100 ± 1.39 | 1.377 ± 0.016 | 100 ± 1.19 |
| 0.0512 | 1.448 ± 0.015 | 103.64 ± 1.10 | 1.445 ± 0.032 | 104.99 ± 2.33 |
| 0.25 | 1.455 ± 0.013 | 104.18 ± 0.96 | 1.461 ± 0.021 | 106.09 ± 1.53 |
| 1.28 | 1.487 ± 0.005 | 106.44 ± 0.38 | 1.496 ± 0.013 | 108.69 ± 0.91 |
| 6.4 | 1.487 ± 0.024 | 106.42 ± 1.72 | 1.433 ± 0.015 | 104.1 ± 1.1 |
| 32 | 1.455 ± 0.024 | 104.15 ± 1.74 | 1.291 ± 0.024 | 93.78 ± 1.75 |
| 160 | 1.337 ± 0.018 | 95.72 ± 1.31 | 1.175 ± 0.02 | 85.31 ± 1.43 |
| 800 | 1.255 ± 0.031 | 89.87 ± 2.19 | 1.160 ± 0.03 | 84.26 ± 2.19 |
| 4,000 | 1.199 ± 0.019 | 85.83 ± 1.34 | 1.171 ± 0.021 | 85.04 ± 1.55 |
| 20,000 | 1.213 ± 0.013 | 86.80 ± 0.96 | 1.156 ± 0.022 | 83.96 ± 1.61 |
| 100,000 | 1.213 ± 0.038 | 86.80 ± 2.69 | 1.131 ± 0.017 | 82.15 ± 1.27 |

TABLE 4

| | 3 days | | 6 days | |
|---|---|---|---|---|
| Concentration | | Cell | | Cell |
| (IU/ml) | OD | viability (%) | OD | viability (%) |
| 0 (medium | 0.715 ± 0.013 | 100 ± 1.770 | 1.945 ± 0.092 | 100 ± 4.750 |
| control group) | | | | |
| 0 (solvent | 0.650 ± 0.037 | 90.977 ± 5.221 | 1.728 ± 0.039 | 88.86 ± 2.002 |
| control group) | | | | |
| 390 | 0.653 ± 0.024 | 91.397 ± 3.289 | 2.016 ± 0.162 | 103.676 ± 8.342 |
| 780 | 0.619 ± 0.019 | 86.605 ± 2.653 | 1.939 ± 0.025 | 99.717 ± 1.264 |
| 1,560 | 0.614 ± 0.028 | 85.941 ± 3.934 | 1.894 ± 0.159 | 97.391 ± 8.157 |
| 3,130 | 0.498 ± 0.026 | 69.643 ± 3.706 | 1.374 ± 0.014 | 70.627 ± 0.714 |
| 6,250 | 0.452 ± 0.018 | 63.208 ± 2.576 | 1.142 ± 0.114 | 58.711 ± 5.885 |
| 12,500 | 0.472 ± 0.073 | 66.006 ± 10.174 | 0.716 ± 0.021 | 36.820 ± 1.080 |
| 25,000 | 0.397 ± 0.038 | 55.514 ± 5.282 | 0.606 ± 0.016 | 31.164 ± 0.827 |
| 50,000 | 0.397 ± 0.055 | 55.584 ± 7.673 | 0.608 ± 0.039 | 31.279 ± 1.957 |

What is claimed is:

1. A low-dose interferon-containing composition, for treatment or prevention of conditions, diseases or disorders associated with inflammation in cat, wherein the composition comprises a total concentration of human interferon-α2b in a range of 160 IU/ml to 100,000 IU/ml.

2. The composition of claim 1, wherein the composition further includes one or more additives, which include cellulose, starch, corn starch, sugar, lactose, glucose, sorbitol, mannitol, maltose powder, malic acid, silicon dioxide or any combination thereof.

3. The composition of claim 1, wherein the composition further includes a maltose powder, a silicon dioxide and a malic acid, a weight of the maltose powder accounts for a total weight of the composition between 90.00% and 99.50%, a weight of the silicon dioxide accounts for the total weight of the composition is between 0.1% to 2.0%, and a weight of the malic acid accounts for the total weight of the composition is between 0.01% and 9.50%.

4. The composition of claim 1, wherein the composition is orally administered buccally.

5. The composition of claim 1, wherein an administration of the composition is once-daily.

6. The composition of claim 1, wherein the composition is a food composition or a pharmaceutical composition.

7. A use of the composition according to claim 1 in a manufacture of a medicament for the treatment or prevention of conditions, diseases or disorders associated with inflammation in cat.

8. The use of claim 7, wherein the composition is orally administered buccally.

9. The use of claim 7, wherein an administration of the composition is once-daily.

10. A method for treatment or prevention of conditions, diseases or disorders associated with inflammation in cat, comprising:
orally administering to a cat subject of a low-dose interferon-containing composition in a daily dose of 160 IU/ml to 100,000 IU/ml human interferon-α2b.

11. The method of claim 10, wherein the cat subject experiences low adverse effects after administration.
